(19) United States
(12) Patent Application Publication
(10) Pub. No.: US 2014/0071238 A1
(43) Pub. Date: Mar. 13, 2014

(54) DEVICES AND METHODS FOR VISUALIZATION AND THREE-DIMENSIONAL RECONSTRUCTION IN ENDOSCOPY

(71) Applicants: Benjamin Mertens, Uccle (BE); Pascal Kockaert, Etterbeek (BE)

(72) Inventors: Benjamin Mertens, Uccle (BE); Pascal Kockaert, Etterbeek (BE)

(21) Appl. No.: 14/080,584

(22) Filed: Nov. 14, 2013

Related U.S. Application Data
(63) Continuation of application No. PCT/EP2012/059023, filed on May 15, 2012.

(30) Foreign Application Priority Data
May 16, 2011 (EP) 11166180.7

(51) Int. Cl. A61B 1/00

(52) U.S. Cl. CPC A61B 1/00193; A61B 1/00101; A61B 1/00087
USPC 348/45

(57) ABSTRACT

Example devices and methods for visualization and three-dimensional reconstruction of an area of interest are disclosed. An example device includes a first light source able to send quasi-monochromatic light through a monomode optical fibre and a second light source able to send light through a set of optical fibres. The example device also includes a diffractive element that induces a pattern to be projected on an area of interest when the first light source is switched on. In addition, the example device includes a camera that has a spatiotemporal resolution such that it is able to visualize the pattern created by the first light source and the area of interest illuminated by the second light source that appears uniformly illuminated even if diffractive element covers at least partially the second cross-section of the set of optical fibres.

### RELATED APPLICATIONS

This patent is a continuation of International Patent Application Serial No. PCT/EP2012/059023, filed May 15, 2012, which claims priority to European Patent Application 11166180.7, filed on May 16, 2011, both of which are hereby incorporated herein by reference in their entireties.

### FIELD OF DISCLOSURE

This disclosure relates generally to the field of endoscopy. More specifically, this disclosure relates to devices and methods for visualization and three-dimensional reconstruction of an area of interest.

### BACKGROUND

Endoscopy allows clinicians to visualize internal organs to screen for diseases such as colorectal or oesophagus cancers for instance. As discussed in US2007/0197862, an endoscope can be coupled with chirurgical tools (typically jointed arms) allowing local surgery with less invasive impacts than conventional surgery. Endoscopes allow illumination of an area of interest and its visualization with a camera. Regular video cameras do not allow a clinician to position surgical tools in space since a third dimension is required. Therefore, clinicians want endoscopes equipped with a minimally invasive three-dimensional viewing system or three-dimensional reconstruction system. Three-dimensional reconstruction of an area of interest can be performed by analyzing a deformation of a known pattern when it is sent on the area of interest.

Examples of endoscopes allowing visualization and three-dimensional reconstruction of an area of interest are described in U.S. Patent Publication US2010/0149315 and in Chinese Publication CN201429412. The device described in CN201429412 comprises a laser projection system and an illumination system. The laser projection system comprises a laser that sends coherent light through a monomode optical fibre to a diffraction grating (or diffractive element) positioned at a distal end of the endoscope. This results in the formation of a pattern on an area of interest. By analyzing the deformation of this pattern on the area of interest, one can perform its three-dimensional reconstruction. The illumination system comprises a Light-Emitting Diode (LED) positioned at a distal end of the endoscope that illuminates the area of interest through a set of lenses. A camera positioned at same distal end allows visualizing the pattern and the area of interest illuminated by the LED photo source. The device described in CN201429412 thus allows visualization and three-dimensional reconstruction of an area of interest but requires a rather deep change with respect to common endoscopes.

FIG. 1 of US2010/0149315 shows an example of endoscope including an imaging channel, an illumination channel, and a projection channel. A CCD camera is used to capture images through the imaging channel. Through the projection channel, a collimated light source from a laser diode and a holographic grating are used to generate structured light. By analyzing the deformation of the structured light on an area of interest, its three-dimensional reconstruction can be performed. White light source is used for illuminating the area of interest. A drawback of a system such as the one shown in US2010/0149315 is that it is not compact enough and that it is relatively difficult to fabricate.

### SUMMARY

It is an object of the present disclosure to provide a device for visualization and three-dimensional reconstruction of an area of interest that is more compact and that is easier to fabricate. To this end, an example the device disclosed herein includes a tubular shell having a proximal end and a distal end. The example device also includes a pattern projection optical group that includes a first light source that is quasi-monochromatic and at least one monomode optical fibre positioned in the tubular shell, having a first extremity, a second extremity, and a first cross-section, and able to transport light through the first cross-section. In this example, the first extremity lies at the proximal end, and the second extremity lies at the distal end. The example pattern projection optical group also includes a first optical path between the first light source and the first extremity. The example device also includes an illumination optical group that includes a second light source and a set of optical fibres positioned in the tubular shell. In this example, the set of optical fibres has a third and a fourth extremity and a second cross-section. The third extremity lies at the proximal end, and the fourth extremity lies at the distal end. The example illumination optical group also includes a second optical path between the second light source and the third extremity. In addition, the example device includes a diffractive element covering the first cross-section at the distal end and a camera having a spatiotemporal resolution. Furthermore, in the disclosed example, the at least one monomode optical fibre and the set of optical fibres are included in a same optical fibre bundle of outer diameter D_{bundle}. In addition, the diffractive element covers at least partially the second cross-section of the set of optical fibres at the fourth extremity. Also, in the disclosed example, the spatiotemporal resolution of the camera is such that the camera is able to provide an image of a pattern created by the pattern projection optical group and the diffractive element on the area of interest, and able to provide a two-dimensional image of the area of interest created by the illumination optical group that appears uniformly illuminated. Different examples of the spatiotemporal resolution of the camera allowing it to provide such images are provided herein. Stating that the camera is able to provide a two-dimensional image of the area of interest created by the illumination optical group that appears ‘uniformly illuminated’ means that the camera is unable to provide images of a pattern (or of any interference phenomena) created by the illumination optical group.

In an example device of the present disclosure, the first light source is quasi-monochromatic. As light arising from a monomode optical fibre has a small spatial extension in plane perpendicular to a direction of light propagation and as the diffractive element covers the first cross-section at the second extremity of the monomode optical fibre, a pattern is formed on the area of interest when the first light source is switched on. The second light source can send light to the area of interest through the set of optical fibres for illumination purposes. The camera allows providing a first image of a pattern on the area of interest for three-dimensional reconstruction and visualizing the area of interest illuminated by the illumination optical group. As the monomode optical fibre allowing a formation of a pattern on the area of interest is included in a same optical fibre bundle as the set of optical fibres that is used for illumination purposes, one can obtain a device that has a smaller size with respect to the one described in US2010/0149315 or in CN201429412. Contrary to these devices, only one group of optical carriers is used for creating a pattern on the area of interest and for providing an illumination of it that appears uniform. This reduces the size of the device of the present disclosure to one that is more compact.

The first cross-section through which light can be transported in the monomode optical fibre has a small area. The diameter of the first cross-section is indeed comprised between 1 and 10 μm as the first cross-section is a cross-section of a monomode optical fibre through which light is transported. So, providing and fixing a diffractive element that only covers this first cross-section is a constraint that complicates the fabrication of a device for visualization and three-dimensional reconstruction. The examples disclosed herein include the diffractive element that covers at least partially the second cross-section of the set of optical fibres at the fourth extremity. Less precaution is thus required for fabricating the example devices disclosed herein as the diffractive element does not have to only cover the (small) first cross-section. Using a same optical fibre bundle for the monomode optical fibre and for the set of optical fibres also allows facilitating the fabrication of the example devices disclosed herein with respect to other devices.

In a first embodiment of an example device disclosed herein, light exiting the set of optical fibres at the fourth extremity can be quasi-monchromatic (not incoherent) or incoherent. The absence of constraint on the type of light exiting the set of optical fibres at the fourth extremity further facilitates the fabrication of the example devices disclosed herein. When light emerging from the fourth extremity of the set of optical fibres is not incoherent, the camera is able to provide a two-dimensional image of the area of interest created by the illumination optical group that appears uniformly illuminated because of the spatiotemporal resolution of the camera that is specified above. This spatiotemporal resolution of the camera also allows the camera to provide an image of a pattern created by the pattern projection optical group and the diffractive element. Hence, this disclosure details a device for visualization and three-dimensional reconstruction of an area of interest that is more compact and that is easier to fabricate.

The example devices disclosed herein have other advantages. As the example devices disclosed herein are smaller or more compact, these example devices have a higher flexibility thus allowing less invasive, faster and cheaper procedures. Due to the small size, the example devices disclosed herein can also be used in therapeutic techniques of endoscopy where surgical tools are coupled with imaging devices. The use of a diffractive element for three-dimensional reconstruction allows having such a three-dimensional reconstruction in one shot of the camera. Neither scanning techniques nor mirrors mounted on a galvanometer are needed with the example devices. As the second light source is positioned at the proximal end, clinicians can modulate the light properties (its frequency for instance) that is used for illumination in an easier way than if the second light source were positioned at the distal end. Clinicians indeed like to have the possibility to change the properties of light used for illumination depending on the type of tissues that are studied. Endoscopes that are commonly used typically comprise an optical fibre bundle that is used for illuminating an area of interest, see for instance models GIF-H180 from Olympus. For the example devices disclosed herein, one only needs to have one monomode optical fibre from such an optical fibre bundle that is used for carrying quasi-monochromatic light to the diffractive element. No additional light source at the distal end is necessary contrary to the device described in CN201429412 for which a LED is positioned at the distal end. In the examples disclosed herein, an optical fibre bundle is used both for creating a first image of a pattern and a second image of the area of interest that appears uniformly illuminated. Hence, from commonly used endoscopes, one needs to impose less changes with the example devices disclosed herein with respect to the device described in CN201429412. As a consequence, the fabrication of the example devices disclosed herein is easier than the fabrication of the device detailed in CN201429412. As the example devices require fewer changes with respect to common endoscopes and are also more robust (for instance, a higher resistance to corrosion is expected when compared to the device described in CN201429412). The cost of fabrication of the example devices disclosed herein is lower with respect to other devices because the example devices are easier to fabricate.

The structured light is formed at the distal end with the example devices disclosed herein. This allows avoiding deformation of the structured light through the optical fibres contrary to a case where the structured light is formed at the proximal end and carried from proximal end to distal end (as shown in FIG. 21 of US2010/0149315 for instance). The device described in paragraph [0105] of US2010/0149315 is a rigid endoscope. The examples disclosed herein include a device that can be flexible due to its small size, allowing an easier insertion into a cavity to be studied.

Some examples disclosed herein include a device that includes a diffractive element that covers at least 30%, and in some examples, at least 50% or at least 70% of the second cross-section of the set of optical fibres at the fourth extremity. Also, in some examples, the diffractive element totally covers the second cross-section of the set of optical fibres at the fourth extremity. The fabrication of the example device is further facilitated when the diffractive element covers a large part of the second cross-section of the set of optical fibres at the fourth extremity.

In some examples, the illumination optical group is able to provide incoherent light at the fourth extremity. Then, for any spatio-temporal resolution of the camera, a two-dimensional image of the area of interest created by the illumination optical group appears uniformly illuminated. Incoherent light passing through a diffractive element cannot indeed create a pattern on an area of interest.

In some examples, the camera has an outer diameter A_{cam} such that A_{cam}<2.4 D_{bundle}. Then, if quasi-monochromatic light is provided at the fourth extremity by two external fibres of the set of optical fibres, the condition that the camera provides a two-dimensional image of the area of interest that appears uniformly illuminated is automatically satisfied. The parameter A_{cam} can also be the outer diameter of a lens of the camera or the outer diameter of a diaphragm. In some examples, this outer diameter A_{cam} is adjustable.

In some examples, the camera has an outer diameter A_{cam} such that A_{cam}<0.6 D_{bundle}. Then, if quasi-monochromatic light is provided at the fourth extremity by all the optical fibres of the set of optical fibres, the condition that the camera provides a two-dimensional image of the area of interest that appears uniformly illuminated is automatically satisfied. This condition on the outer diameter of the camera results from statistical calculations that are mentioned in the detailed description. The parameter A_{cam} can also be the outer diameter of a lens of the camera.

In some examples, the area of interest has an outer diameter equal to φ, the camera and the fourth extremity of the set of optical fibres are positioned at a same distance L from the area of interest, the second light source is a quasi-monochromatic light source having a central wavelength equal to λ, and the camera has a number of pixels along one direction, Nₗ, such that Nₗ<2φ/L D_{bundle}/λ. Then, if quasi-monochromatic light is provided by all the optical fibres of the set of optical fibres, the camera provides a two-dimensional image of the area of interest that appears uniformly illuminated.

In some examples, the camera is positioned at the distal end. In some examples, the camera is positioned at the proximal end. In this case, dedicated channels such as optical fibres are typically used for carrying images from the distal end to the camera through the optical fibre bundle. Such an example has the advantage of allowing a use of the device for studying critical or dangerous environments. An example of a dangerous environment is a cavity comprising gases that can easily explode and/or burst in flames. For such environments, it is desired not to introduce electrical components that can induce an explosion or a fire of such gases. Another advantage of using a camera positioned at the proximal end is that frequency multiplexing is then more easily implemented as one can easily change filters positioned before the camera.

In some examples, the pattern projection optical group and the diffractive element are able to provide an uncorrelated pattern on the area of interest. As the pattern projection optical group and the diffractive element are able to provide an uncorrelated pattern on the area of interest, its three-dimensional reconstruction is facilitated. Different parts of the pattern are then unique and are thus easily identified. Of course, other methods are possible, see for instance Salvi et al., “A state of the art in structured light patterns for surface profilometry”, in Pattern recognition, 43 (2010), 2666-2680.

In some examples, multiplexing is used for distinguishing a first image of a pattern created by the pattern projection optical group and the diffractive element from a second image created by the illumination optical group. Also, in some examples, the multiplexing is a temporal multiplexing inducing light to be emitted from the first light source in a pulsed manner. Such examples allow one to distinguish the pattern from pictures visualized by a user. A pattern could indeed disturb a user of the example device. In these examples, the image shown to a user is filtered from the pattern and a processing unit records the pattern and processes the pattern. When temporal multiplexing is used, the first light source is pulsed during short time frames and the processing unit only shows the user the image when this first light source is off (unless the time frame is short enough). In another example, spectral multiplexing is used: a specific wavelength is used for the first light source and the pattern is easily extracted from the image visualized by a user.

In some examples, the devices disclosed herein include a set of optical fibres that include multimode optical fibres.

In some examples, the set of optical fibres includes at least a hundred of monomode optical fibres. Also, in some examples, the set of optical fibres includes at least a thousand of monomode optical fibres.

In some examples, the devices disclosed herein include a third optical path between the second light source and the first extremity.

In some examples, the devices disclosed herein include channels in the tubular shell that have a geometry suitable for inserting of tools for manipulating and cutting mammal tissues at the distal end.

In another example disclosed herein is an example device for visualization and three-dimensional reconstruction of an area of interest that includes a tubular shell having a proximal end and a distal end and a pattern projection optical group. In this example, the pattern projection optical group includes a quasi-monochromatic light source and at least one monomode optical fibre positioned in the tubular shell, having a first extremity, a second extremity, and a first cross-section, and able to transport light through the first cross-section. The first extremity lies at the proximal end, and the second extremity lies at the distal end. The example pattern projection optical group also includes a first optical path between the quasi-monochromatic light source and the first extremity. The example device also includes an illumination optical group that has the same quasi-monochromatic light source and a set of optical fibres positioned in the tubular shell. The set of optical fibres has a third and a fourth extremity and a second cross-section. Also, the third extremity lies at the proximal end and the fourth extremity lies at the distal end. In addition, the example illumination optical group includes a second optical path between the quasi-monochromatic light source and the third extremity. The example device also includes a diffractive element covering the first cross-section at the distal end and a camera having a spatiotemporal resolution. In addition, in this example, the at least one monomode optical fibre and the set of optical fibres are included in a same optical fibre bundle of outer diameter D_{bundle} Also, in this example, the diffractive element covers at least partially the second cross-section of the set of optical fibres at the fourth extremity. Furthermore, in this example, the spatiotemporal resolution of the camera is such that the camera is able to provide an image of a pattern created by the pattern projection optical group and the diffractive element on the area of interest, and able to provide a two-dimensional image of the area of interest created by the illumination optical group that appears uniformly illuminated. In this example, the cost of fabrication is further reduced and the example device is more compact as there is only one light source.

Also disclosed herein is an example method for visualization and/or three-dimensional reconstruction of an area of interest. The example method includes sending to an area of interest a quasi-monochromatic light through a first cross-section of at least one monomode optical fibre and sending to the area of interest light through a set of optical fibres having a second cross-section. The example method also includes acquiring images of the area of interest by using a camera having a spatiotemporal resolution. In the example method, the at least one monomode optical fibre and the set of optical fibres are included in a same optical fibre bundle of outer diameter D_{bundle} Also, in the example method, a diffractive element covers at least partially the second cross-section of the set of optical fibres. Furthermore, in the example method, the spatiotemporal resolution of the camera is such that the camera is able to provide an image of a pattern created by light emerging from the monomode optical fibre and the diffractive element on the area of interest, and able to provide a two-dimensional image of the area of interest created by light emerging from the set of optical fibres that appears uniformly illuminated. In some examples, the methods disclosed herein include providing surgical tools that are connected to a tubular shell comprising the optical fibre bundle.

### BRIEF DESCRIPTION OF THE DRAWINGS

These and further aspects of the examples disclosed herein will be explained in greater detail by way of example and with reference to the accompanying drawings in which:

FIG. 1 shows an example device according to the teachings of the present disclosure and in relation with a processing unit;

FIG. 2 shows elements of the example device at a proximal part of a tubular shell;

FIG. 3 shows elements of the example device at a distal part of a tubular shell;

FIG. 4 shows a cross-section of an example monomode optical fibre;

FIG. 5 shows reference points of an example pattern projected on an area of interest and their images in a camera;

FIG. 6 shows reference points of an example pattern projected on an area of interest and their images in a camera before and after displacement of an area of interest;

FIG. 7 shows an example device in accordance with the teachings of this disclosure;

FIG. 8 shows elements of the example device at a proximal end of another example device disclosed herein.

The figures are not drawn to scale. Generally, identical components are denoted by the same reference numerals in the figures.

### DETAILED DESCRIPTION

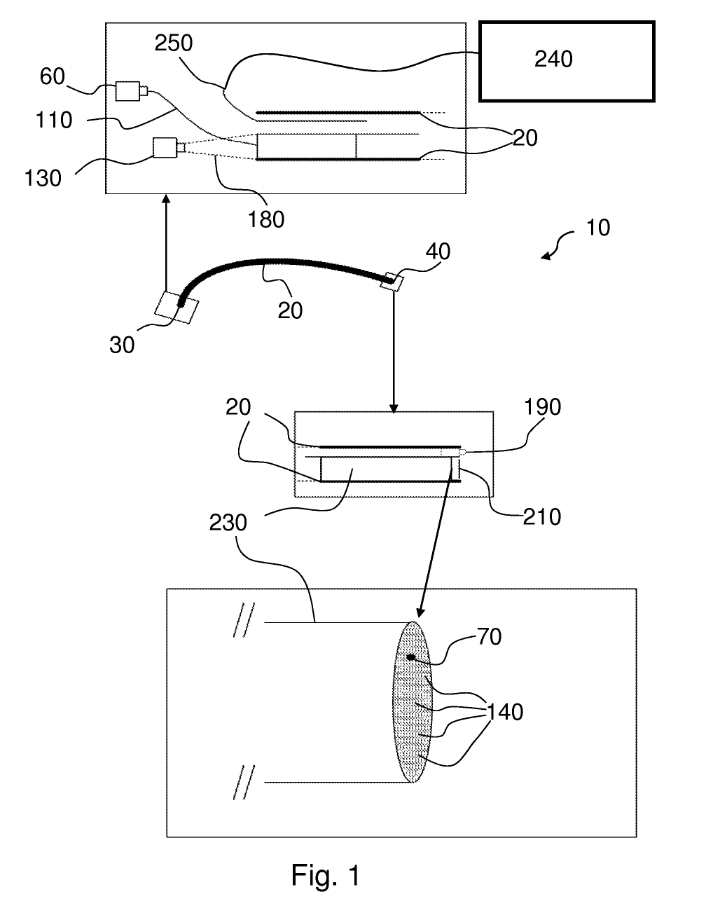

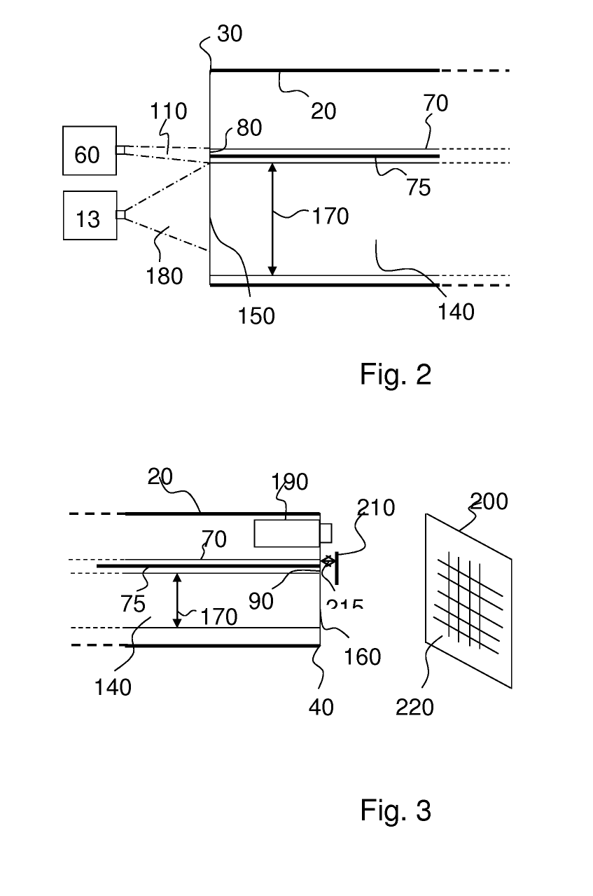

FIG. 1 shows an example device 10 according to the teachings of this disclosure. The example device 10 is shown in relation with a processing unit 240. The device 10 includes a tubular shell 20 having a proximal end 30 and a distal end 40. In some examples, the tubular shell 20 is made of a biocompatible polymer material. The upper part of FIG. 1 is a zoom at the proximal end 30 whereas the lower parts of FIG. 1 detail elements of the example device 10 close to the distal end 40. The elements near the proximal end 30 (respectively distal end 40) are also detailed in FIG. 2 (respectively FIG. 3). The example device 10 also includes a first optical group or pattern projection optical group that comprises a first light source 60 that is quasi-monochromatic, a monomode optical fibre 70 and a first optical path 110 between the first light source 60 and the first extremity 80.

The term quasi-monochromatic is known by the one skilled in the art. Pure monochromatic radiations (or in an equivalent manner pure monochromatic light sources) do not exist physically because of instabilities of light sources or, at an ultimate Fourier limit, because of their finite emission time. Light radiation that behaves like ideal monochromatic radiation is often called quasi-monochromatic. The frequencies of quasi-monochromatic radiations are strongly peaked about a certain frequency. A definition of quasi-monochromatic light source is notably given in “Shaping and Analysis of picoseconds light pulses” by C. Froehly, B. Colombeau, and M. Vampouille in Progress in Optics XX, E. Wolf, North-Holland 1983 (p79). When a space-time light pulse is travelling in the space {x, z}, where z is a coordinate along a direction of propagation of the light pulse, and x states for a coordinate lying in a plane perpendicular to the direction of propagation, the spatial distribution of a light field at any time t can be deduced from the sole knowledge of one of its space time amplitude f_{z}(x,t) at a propagation distance z. For pure monochromatic radiation: f_{z}(x,t)=X_{z}(x)exp{j2πv₀t}, where j is a pure imaginary number. Quasi-monochromatic radiation is usually defined as exhibiting a coherence length larger than the optical path difference involved in a diffracting aperture or interferometer (see for instance Born and Wolf 1965). In a more general case, a quasi-monochromatic light radiation can be defined as: f_{z}(x,t)=m_{z}(x,t) exp{j2πv₀t} where v₀, represents an average frequency of the light radiation and j is an imaginary number. Quasi-monochromatic light will take place only if the space-time modulation m_{z}(x,t) degenerates into a product of a spatial term X_{Z}(x) by a temporal term τ_{z}(t). Then, f_{z}(x,t)=X_{z}(x)τ_{z}(t) exp{j2πv₀t} is a temporal wave train τ_{z}(t) exp{j2πv₀t} modulated by a spatial distribution X_{z}(x). This spatial distribution X_{z}(x) is kept independent on time t at any distance from an origin of light, on the condition that the spectral bandwidth Δv of τ_{z}(t) satisfies a ‘quasi-monochromaticity’ requirement that is Δv<c/δₘₐₓ, c being the speed velocity of light and δₘₐₓ being a maximum optical path difference between outermost rays of such a light beam at a most oblique diffraction angle θ₀ (see FIG. 2.1 p80 of “Shaping and Analysis of picoseconds light pulses” by C. Froehly, B. Colombeau, and M. Vampouille in Progress in Optics XX, E. Wolf, North-Holland 1983). δₘₐₓ may be related to a spatial width Δx and to a highest spatial frequency N₁ of the spatial term X_{z}(x) by the equation: δₘₐₓ=Δx sin θ₀=ΔxN₁c/v₀. Δx represents a spatial extension of a light source or a spatial extension of a light beam passing through a diffractive element as an example. Then, a condition for a ‘quasi-monochromatic’ light source is given by equation (Eq. 1):

$$ℱ = \frac{{v}_{0}}{Δ v} > {N}_{1} Δ x . \qquad ( Eq . 1 )$$

The ratio

$$ℱ = \frac{{v}_{0}}{Δ v}$$

is named spectral finesse. N₁ is an upper limit of the space frequency spectrum F_{z}(Nₓ) of X_{z}(x) (or a highest spatial frequency of F_{z}(Nₓ)) where:

F_{z}(Nₓ)=∫_{-∞}^{+∞}X_{z}(x)exp(−j2πNₓx)dx.

In practice, N₁ is determined by a spatial frequency spectrum of light that is sent and a particular structure of a diffractive element. As a summary, a quasi-monochromatic light source or quasi-monochromatic light is here considered as a light source or light for which

$$ℱ = \frac{{v}_{0}}{Δ v} > {N}_{1} Δ x \qquad ( Eq . 1 )$$

In the opposite, incoherent light or incoherent light source is here given by (Eq. 2):

$$ℱ = \frac{{v}_{0}}{Δ v} \leq {N}_{1} Δ x . \qquad ( Eq . 2 )$$

Equations (Eq. 1) and (Eq. 2) are valid when only one transverse dimension x is considered. In practice, for a typical diffractive element 210, one has to consider two transverse dimensions, x and y. Then, X_{z}(x) becomes X_{z}(x,y). Two examples of quasi-monochromatic light source are a laser and a time-modulated laser for which Δv increases but can be kept limited. Another possibility to have quasi-monochromatic light is to have light with a weak spectral dispersion and that originates from a single point source (for instance at the output of a monomode optical fibre) for which N₁Δx=1. Indeed, light exiting a monomode optical fibre is perfectly Gaussian. In such a case, one needs to have [symbol] >1 which is easily satisfied. Another possibility to have quasi-monochromatic light is to have Nₛₑₜ monomode optical fibres that are included in an optical fibre bundle having a diameter equal to D_{bundle} and that transport light from a quasi-monochromatic light source and assuming that phase shift is induced along the different monomodes optical fibres. Then, light exiting the set of such monomode optical fibres is quasi-monochromatic if [symbol] >D_{bundle}/Nₛₑₜ.

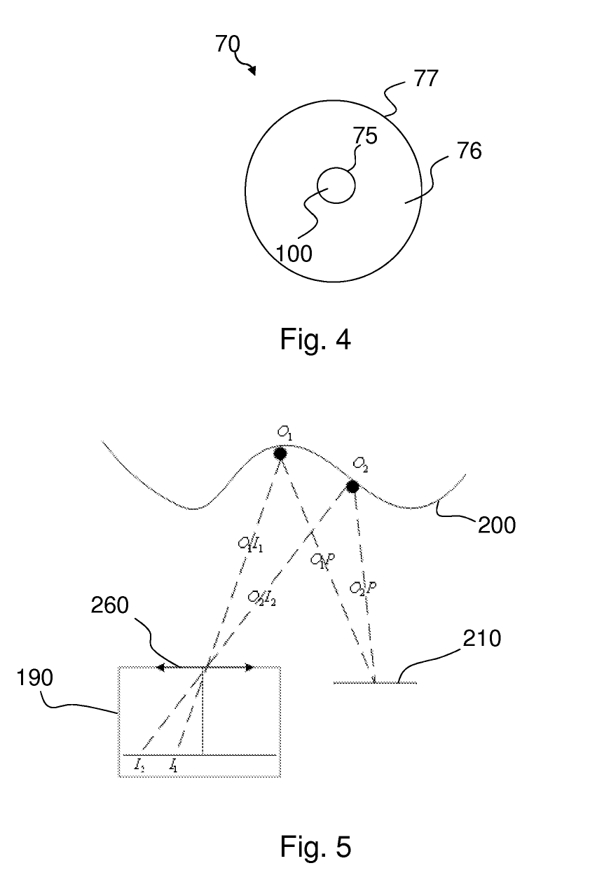

Optical fibres are well known by the one skilled in the art. FIG. 4 shows a cross-section of an exemplary monomode 70 step index or gradient index optical fibre. An optical fibre is a thin and flexible light guide (or wave guide). In some examples optical fibres are made of silica, in some examples optical fibres are cylindrical, and in some examples optical fibres are composed of three layers having different refractive indices (see for instance B Chomycz in “Fiber optic installer's field manual” Mc Graw-Hill 2000). The example device 10 can use other types of optical fibres than step index or gradient index optical fibers. Other examples of optical fibers are microstructured optical fibers. Two types of optical fibres are generally defined: monomode optical fibers 70 and multimode optical fibers. In a general case, a monomode optical fiber is characterized by V<2.4 where V is a reduced frequency. A definition of the reduced frequency V is given by equation (1) of the article entitled “Endlessly single-mode photonic crystal fiber” by T. A. Birks, J. C. Knight, and P. St. J. Russell and published in OPTICS LETTERS Vol. 22, No. 13, Jul. 1, 1997 for step index optical fibers and by equation (6) of the same article for more complex structures such as microstructured optical fibers. In the example of FIG. 4, a core 75 carries light along a longitudinal length of the optical fibre, a cladding layer 76 confines light in the core 75, and a coating layer 77 protects the cladding layer 76 and the core 75. When optical fibres are included in an optical fibre bundle 230, each optical fibre generally does not comprise a coating layer 77. Then, such a coating layer 77 is positioned on an external surface of the optical fibre bundle 230. Light guides (or optical fibres) can propagate light according to different modes of propagation as known by the one skilled in the art. Monomode optical fibres propagate light according to a single mode (or main mode). When working with visible light, monomode optical fibres such as the one of FIG. 4 (step index optical fiber) typically have a core having a diameter equal to or smaller than 10 μm. In some examples, the diameter of the core 75 of such a monomode optical fibre is comprised between 1 and 10 μm. Also, in some examples, the diameter of the core 75 of such a monomode optical fibre is equal to 8 μm. Optical fibres are able to transport light through a first cross-section 100. In the case of step index optical fibres, this first cross-section 100 is the cross-section of the core 75 as shown in FIG. 4. The monomode optical fibre 70 of the example device 10 is positioned in a tubular shell 20, has a first 80 and second 90 extremity. Quasi-monochromatic light may be obtained by using light exiting a monomode optical fiber with a limited Δv because light exiting a monomode optical fibre 70 is a Gaussian beam for which quasi-monochromaticity is easily verified (equation (Eq. 1) then reduces to [symbol] >1).

As shown in the upper part of FIG. 1 and in FIG. 2, there is a first optical path 110 between the first light source 60 and the first extremity 80 of the monomode optical fibre 70. In some examples, a collimator is used for guiding light arising from the first light source 60 to the first extremity 80 of the monomode optical fibre 70.

The example device 10 also includes a second optical group or an illumination optical group that includes a second light source 130, a set of optical fibres 140 and a second optical path 180. The term set means a plurality, such as, for example, a number larger than 100, and in some examples. a number larger than a thousand. The set of optical fibres 140 is positioned in the tubular shell 20 shown in FIGS. 1 to 3. It has a third 150 and a fourth 160 extremity. The second optical path 180 allows light produced by the second light source 130 to be carried to the third extremity 150. In some examples, lenses are used to guide light from the second light source 130 to the third extremity 150.

The monomode optical fibre 70 and the set of optical fibres 140 are part of a same optical fibre bundle 230 as shown in the lower part of FIG. 1. Particular examples are shown in FIGS. 2 and 3 where the monomode optical fibre 70 is a step index optical fibre and adjacent to the set of optical fibres 140 (these two figures are not drawn to scale). An optical fibre bundle 230 is a term known by the one skilled in the art and typically comprises a hundred or more optical fibres. Optical fibres that are used for illumination are typically wrapped in optical fibre bundles 230 so they can be used to carry light in tight spaces. Optical fibre bundles 230 are used in endoscopy to illuminate an area of interest 200. The model IGN 037/10 from Sumitomo Electric of optical fibre bundle 230 comprises 10 000 optical fibres. In an example where the set of optical fibres 140 includes monomode optical fibres, a monomode optical fibre bundle 230 that is commercially available may be used. One of the optical fibres is selected for transporting light emitted by the first light source 60. Optical fibre bundles 230 have a cross-section whose diameter is, in some examples, between about 0.5 and about 10 mm, and in some examples around 1 mm.

The example device 10 also includes a diffractive element 210 (or diffraction grating) covering the first cross-section 100 at the second extremity 90 and covering at least partially the second cross-section 170 of the set of optical fibres 140 at the fourth extremity 160. The diffractive element 210 is positioned at a certain small distance 215 from the second extremity 90. In some examples, the distance 215 is larger than \sqrt{a²−w₀²}/θ₀, where a is a dimension of the diffractive element 210 (e.g., the radius measured perpendicular to the direction of propagation of light), w₀ is related to a width of a mode of propagation of a light beam exiting the monomode optical fibre 70, and θ₀=λ₀/(πw₀) where λ₀ is a mean frequency of a quasi-monochromatic light. In some examples, this distance 215 is equal to several multiples of the mean wavelength λ₀ of the light emitted by the first light source 60. In some examples, this distance 215 is between about 100 nm and about 1800 nm. Light arising from the second extremity 90 of the monomode optical fibre 70 has to pass through the diffractive element 210 before hitting an area of interest 200. A diffractive element 210 is an optical component with a structure that splits and diffracts light into several beams. The diffractive element 210 is used for producing a pattern 220 on an area of interest 200 with light arising from the second extremity 90 of the monomode optical fibre 70. For producing such a pattern 220, an example of a diffractive element 210 comprises a set of grooves or slits that are spaced by a constant step d. In some examples, such a diffractive element 210 includes grooves that are parallel to two directions perpendicular to a direction of propagation of light originating from the second extremity 90. To obtain an observable pattern 220, a step d is used between the grooves that is of the same order of magnitude as the mean wavelength λ₀ of the first light source 60 that is quasi-monochromatic. That means that in some examples λ₀/10<d<10λ₀. Also, in some examples, the step d is between about 10 nm and about 25000 nm, and in some examples, equal to 400 nm. Also, in some examples, the diffractive element 210 includes regions with various thicknesses that induce local phase variations of a beam light passing through it. With the example device 10, a pattern 220 can be obtained because light arising from the second extremity 90 and passing through the diffractive element 210 is quasi-monochromatic. Other types of diffractive elements 210 can be used. In some examples, holographic gratings can be used for which rather complicated patterns 220 can be obtained. The pattern 220 can take a variety of forms including stripes, grids, and dots as an example.

The example device 10 also includes a camera 190. In the examples shown in FIG. 1 and FIG. 3, the camera 190 is positioned at the distal end 40 in the tubular shell 20. This camera 190 is able to provide dynamic two-dimensional pictures of an area of interest 200 illuminated by the illumination optical group through the fourth 160 extremity (what we name second images), the two-dimensional pictures appearing uniformly illuminated. The camera 190 is also able to provide dynamic pictures of the pattern 220 created by the pattern projection optical group and the diffractive element 210 and projected on the area of interest 200 (e.g., first images). Various types of camera 190 (such as CCD cameras) that are used for endoscopy can be used for the example device 10. An example of such a camera 190 is a cylindrical camera named VideoScout sold by BC Tech (a medical product company) that has a diameter of 3 mm but commonly used camera in endoscopy are suitable. The tubular shell 20 of the example device 10 has a diameter ranging between about 4 mm and about 2 cm. The camera 190 is connected to a processing unit 240 through cables 250. In some examples, the illumination optical group provides light that is not incoherent at the fourth extremity 160. That is notably the case when the second light source 130 is quasi-monochromatic and when the set of optical fibres 140 comprise monomode optical fibres for which [symbol] >D_{bundle}/Nₛₑₜ, where D_{bundle} is the outer diameter of the optical fibre bundle 230 and where Nₛₑₜ is the number of optical fibres in the set of optical fibres 140. For such an example, even if the diffractive element 210 covers at least partially the second cross-section 170 of the set of optical fibres 140 at the fourth extremity 160, the camera 190 is able to provide a two-dimensional image of the area of interest 200 created by the illumination optical group that appears uniformly illuminated. This is possible thanks to the spatiotemporal resolution of the camera 190 for which different possible examples are given below. If light provided by the illumination optical group induces interference phenomena, such phenomena are indeed unobservable by a camera if its spatiotemporal resolution is not adapted for detecting them. It then follows that a uniformly illuminated image (second image) is provided by the camera 190. The spatiotemporal resolution of the camera 190 is nevertheless such that the camera 190 is able to provide an image of a pattern 220 created by the pattern projection optical group and the diffractive element 210. Such a property is readily satisfied for cameras 190 that are commonly used in the field of endoscopy as it is shown below with an illustrative example.

The example pattern 220 includes 64 lines, and the first light source 60 is a quasi-monochromatic light source having a central wavelength equal to λ. The angle of incidence is zero with respect to an axis that is normal to the diffractive element 210. Then, the maximum angle of diffraction, βₘₐₓ, for the 32th orders of diffraction is given by sin βₘₐₓ=32λ/d, where d is the step between grooves or slits of the diffractive element 210 (this expression can be easily found from the lax of diffraction induced by a network comprising grooves). Such an order of diffraction is only visible if 32λ/d≦1 which means d≧32λ. The spatiotemporal resolution of the camera 190 must be such that two successive orders of diffraction are distinguishable. If δβ represents the angle difference between the angles of diffraction of K and K−1 orders, one can show that δβ≈λ/d cos β_{K} (β_{K} is the angle of diffraction of order K). The minimal spatiotemporal resolution is reached when cos β_{K}=1, for which δβ≈λ/d≦1/32 from the previous calculation. The optical resolution of the camera 190 is given by r_{θ}1.2λ/A_{cam}, where A_{cam} is the outer diameter of the camera 190. Then imposing that the spatiotemporal resolution of the camera 190 is such that it is able to distinguish between two lines of the pattern 220, and the following condition is satisfied:

1.2λ/A_{cam}=0.5λ/d<1/64

or

A_{cam}=2.4d>2.4*32λ=38.4 μm

if λ=500 nm. Such a condition is readily satisfied for cameras 190 commonly used in endoscopy. The minimum number of pixels of the camera 190 is 128. This last condition is also satisfied. In some examples, a camera 190 having 500 pixels and an outer diameter, A_{cam}, equal to 3 mm is used.

The example processing unit 240 includes a board such as a frame grabber for collecting data from the camera 190. The processing unit 240 can be an ordinary, single processor personal computer that includes an internal memory for storing computer program instructions. The internal memory includes both a volatile and a non-volatile portion. Those skilled in the art will recognize that the internal memory can be supplemented with computer memory media, such as compact disk, flash memory cards, magnetic disc drives.

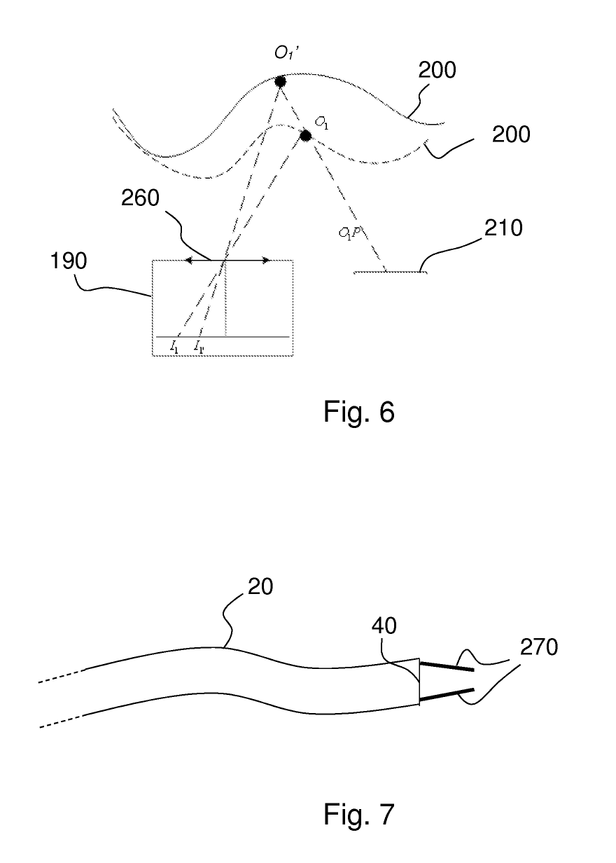

The example device 10 uses a technique named structured light analysis or active stereo vision for three-dimensional reconstruction of an area of interest 200 (see for instance the article by T T W J Y Qu entitled “Optical imaging for medical diagnosis based on active stereo vision and motion tracking” in Opt. Express, 15: 10421-10426, 2007). Three-dimensional reconstruction refers to a generation of three-dimensional coordinates representing an area of interest 200. The example device 10 allows measuring different distances or dimensions, thus providing quantitative information. Another term for three-dimensional reconstruction is three-dimensional map. Structured light analysis allows three-dimensional reconstruction of an area of interest 200 by analyzing a deformation of a pattern 220 when it is projected on an area of interest 200. For explaining this technique, assume that the pattern 220 is a grid as shown in FIG. 3. Then, the intersections of the lines constructing the grid can be used as reference points that are easily located on the area of interest 200. These reference points are named Oᵢ, i=1, 2, . . . , n below. FIG. 5 shows an example of an area of interest 200 on which reference points Oᵢ are projected. Lines OᵢP are defined by the knowledge of the pattern 220 and the position of its source. Indeed, for any pattern 220, it is possible to define a source point P from which the reference points Oᵢ are referred. Such a source point P is typically chosen at the second exit 90 of the monomode optical fibre 70. For a known pattern 220, the angles θᵢ between the lines OᵢP and a reference direction are known. An example of an angle θ₁ is shown in FIG. 5 where the reference direction is horizontal. Iᵢ represent the images of the reference points Oᵢ in the camera 190. In FIG. 5, a lens 260 is shown, this lens 260 focusing an image on a camera sensor. Each reference point Oᵢ represents an intersection between lines OᵢP and OᵢIᵢ. Knowing the distance between the camera 190 and the source point P, the three-dimensional coordinates of the points Oᵢ are found from geometric calculations in triangles formed notably by lines OᵢP and OᵢIᵢ. Such calculations (also named triangulation technique) are known by the one skilled in the art and are typically implemented in a program of the processing unit 240. Details of the method allowing three-dimensional reconstruction are notably presented in US2008/0240502. When the area of interest 200 is displaced, reference points Oᵢ move. FIG. 6 shows an example for a reference point O₁ that is displaced to O₁′ after the displacement of the area of interest 200 (the dashed curve represents the area of interest 200 before displacement). From FIG. 6, we see that the corresponding picture in the camera 190, I₁′, has moved with respect to I₁. By processing the pictures provided by the camera 190, the three-dimensional coordinates of an area of interest 200 can be deduced before and after displacement. In some examples, motion tracking is used for following reference points after a first detection. As known by the one skilled in the art, three-dimensional reconstruction from a triangulation technique needs a calibration phase. Such a calibration phase is explained in the book entitled “Learning OpenCV” by G Bradsky and published by O'Reilly in 2008. Computer software's such as Matlab also propose calibration procedures.

The example device 10 can provide dynamic data, which means three-dimensional reconstruction and two-dimensional pictures of an area of interest 200 dynamically. The example device 10 allows one to observe temporal variations of an area of interest 200. In some examples, the two-dimensional image produced by the illumination optical group is projected on a three-dimensional grid obtained from the three-dimensional reconstruction.

In some examples, the diffractive element 210 covers at least 30%, and in some examples at least 50% or at least 70% of the second cross-section 170 of the set of optical fibres 140 at the fourth extremity 160. Also, in some examples, the diffractive element 210 totally covers the second cross-section 170 of the set of optical fibres 140 at the fourth extremity 160.

In some examples, the illumination optical group is able to provide incoherent light at the fourth extremity 160 of the set of optical fibres 140. That means that light provided by the illumination optical group is such that equation (Eq. 2) is satisfied. There are different possibilities to obtain an illumination optical group able to provide incoherent light at the fourth extremity 160. As an example, a second light source 130 can be used that provides light that is incoherent, for instance a white light source. Another possibility is to use a second light source 130 that is quasi-monochromatic. Then, incoherence (spatial incoherence) at the fourth extremity 160 of the set of optical fibres 140 would result from the propagation of light through the set of optical fibres 140. To obtain incoherent light when using a second light source 130 that is quasi-monochromatic, multimode optical fibres can be used for the set of optical fibres 140. As different modes of propagation exist in such multimode optical fibres, light arising from the fourth extremity 160 is (spatially) incoherent. Step index multimode optical fibres typically have a core 75 whose diameter is larger than about 10 μm, and in some examples, larger than 15 μm. In some examples, more than ten multimode optical fibres are used for the set of optical fibres 140 and in some examples, more than a thousand. In an example where the set of optical fibres 140 includes a large number of monomode optical fibres, such as, for example, a number larger than a hundred and/or larger than a thousand, patterns produced by light originating from the exit of each monomode optical fibre are unpredictable because of deformation of the optical fibre bundle 230, and so unobservable by cameras. As a consequence, light originating from a set of optical fibres 140 including a large number of monomode optical fibres can be used for obtaining a uniformly illuminated image of the area of interest 200 with commonly used cameras.

In some examples, the camera 190 has an outer diameter A_{cam} such that A_{cam}<2.4 D_{bundle}, where D_{bundle} is the outer diameter of the optical fibre bundle 230. Theoretically, if the camera 190 has an outer diameter equal to A_{cam} and if the optical fibre bundle 230 has an outer diameter equal to D_{bundle}, light emitted by two monomode optical fibres of the optical fibre bundle 230 that are separated by D_{bundle} leads to a second picture seen by the camera 190 that appears uniformly illuminated if A_{cam}<2.4 D_{bundle}, even if the second light source 130 is quasi-monochromatic. Hence, when A_{cam}<2.4 D_{bundle} and when light at the fourth extremity 160 is provided by two monomode optical fibres that are separated by D_{bundle}, the condition that the camera 190 is able to provide a two-dimensional image of the area of interest 200 created by the illumination optical group that appears uniformly illuminated is automatically satisfied, even if the second light source 130 is quasi-monochromatic, and even if the diffractive element 210 covers at least partially the second cross-section 170

In some examples, the camera 190 has an outer diameter A_{cam} such that A_{cam}<0.6 D_{bundle}. When this condition is satisfied, and when all the optical fibres of the set of optical fibres 140 are monomode optical fibres that transport light from a second light source 130 that is quasi-monochromatic, the condition that the camera 190 is able to provide a two-dimensional image of the area of interest 200 created by the illumination optical group that appears uniformly illuminated is automatically satisfied (even if the diffractive element 210 covers at least partially the second cross-section 170). Such a condition, A_{cam}<0.6 D_{bundle}, can be deduced from theoretical calculations based on the approach followed in the article by T. L. Alexander et al., entitled “Average speckle size as a function of intensity threshold level: comparison of experimental measurements with theory”, published in Applied Optics, Vol. 33, No. 35, in 1994 (p8240). This approach uses the speckle theory.

In some examples, a diaphragm is introduced between the camera 190 and the area of interest 200 in order to reduce the effective parameter A_{cam} entering the above equations (in such a case, A_{cam} is not the actual outer diameter of the camera 190 but rather the aperture of the diaphragm).

In some examples, the camera 190 has a number of pixels along one direction, Nₗ, such that

$${N}_{l} < 2 \frac{φ}{L} \frac{{D}_{bundle}}{λ} .$$

This last formula is based on the assumptions that the camera 190 and the fourth extremity 160 of the set of optical fibres 140 are positioned at a same distance L from the area of interest 200, and that second light source 130 is a quasi-monochromatic light source having a central wavelength equal to λ. Parameter φ is the outer diameter of the area of interest 200 (or the size of the largest side of the area of interest 200 if the area of interest 200 has a rectangular shape). When the condition

$${N}_{l} < 2 \frac{φ}{L} \frac{{D}_{bundle}}{λ}$$

is satisfied, and when all the optical fibres of the set of optical fibres 140 are monomode optical fibres that transport light from a second light source 130 that is quasi-monochromatic, the condition that the camera 190 is able to provide a two-dimensional image of the area of interest 200 created by the illumination optical group that appears uniformly illuminated is automatically satisfied (even if the diffractive element 210 covers at least partially the second cross-section 170). Such a condition can also be found from theoretical calculations based on the approach developed by T. L. Alexander et al., in “Average speckle size as a function of intensity threshold level: comparison of experimental measurements with theory”, Applied Optics, Vol. 33, No. 35, in 1994 (p8240). If φ=2 cm, L=6 cm, D_{bundle}=1 mm, and λ=500 nm, then in this example, Nₗ<667. Such a condition is easily satisfied with cameras 190 commonly used in endoscopy.

In some examples, the camera 190 is positioned at the proximal end 30 of the tubular shell 20. Then, means (e.g., optical fibres) allow light of the pattern and light of the area of interest illuminated by the illumination optical group to be transported to the camera 190 through the tubular shell 20. In another example, the camera 190 is positioned at the distal end 30 of the tubular shell 20. In some examples, the second light source 130 is a source of white light.

In some examples, the first light source 60 is a laser.

In some examples, the pattern projection optical group and the diffractive element are able to provide an uncorrelated pattern on the area of interest 200. An uncorrelated pattern of spots is explained in US2008/0240502. The term uncorrelated pattern refers to a pattern 220 of spots whose positions are uncorrelated in planes transverse to a projection beam axis (from the second extremity 90 to the area of interest 200). In some examples, the pattern 220 is pseudo random which means that the pattern 220 is characterized by distinct peaks in a frequency domain (reciprocal space), but contains no unit cell that repeats over an area of the pattern 220 in a spatial domain (real space). In some examples, a lens is inserted between the second extremity 90 of the monomode optical fibre 70 and the diffractive element 210.

In some examples, multiplexing is used for distinguishing the pattern 220 from the images shown to a user by the camera 190. This provides to a user a more comfortable visualization of an area of interest 200 (the shown pictures are filtered from the pattern 220). In parallel, the processing unit 240 performs three-dimensional reconstruction from the acquisition of the deformation of the pattern 220 on the area of interest 200. Two examples of multiplexing are spectral and temporal multiplexing. In the first case, a specific mean wavelength is used for the quasi-monochromatic first light source 60. This facilitates extraction of the pattern 220 from the pictures shown to a user. When temporal multiplexing is used, the first light source 60 emits light in a pulsed manner during short time frames. If such frames are short enough, the pattern 220 cannot be observed by a user. Otherwise, the processing unit 240 only shows to a user pictures when the first light source 60 is switched off. Temporal multiplexing can also be used for removing images produced by the light provided by the illumination optical group when analyzing the pattern for three-dimensional reconstruction. This allows a higher contrast of the pattern 220.

In some examples, the example device 10 further includes a third optical path between the second light source 130 and the first extremity 80 of the monomode optical fibre 70. In this example, the monomode optical fibre 70 transports light both from the first 60 and second 130 light source.

FIG. 7 shows a part of another example of the device 10. In this example, the device 10 further includes channels in the tubular shell 20 allowing insertion of tools such as, for example, jointed arms 270 for manipulating and/or cutting mammal tissues at the distal end 40. These channels can also be used for water injection.

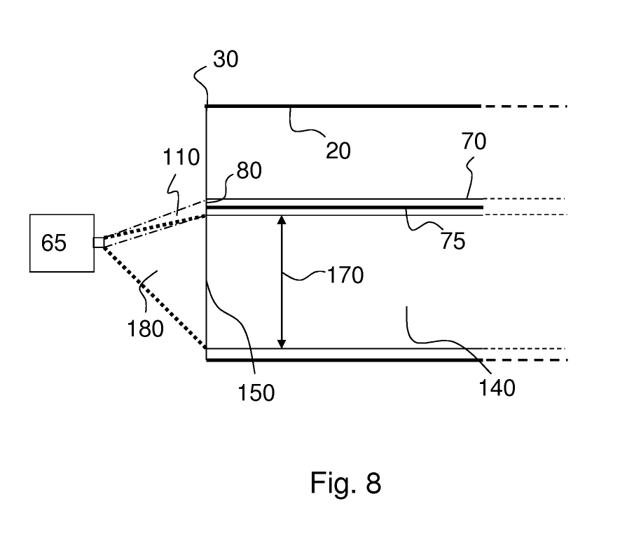

In some examples, the first 60 and second 130 light sources are identical and are a same quasi-monochromatic light source 65. The proximal end of this example is shown in FIG. 8. The first optical path 110 allows a transmission of light from the quasi-monochromatic light source 65 to the monomode optical fibre 70 whereas the second optical path 180 allows a transmission of light from the quasi-monochromatic light source 65 to the set of optical fibres 140. Such example allows obtaining a still more compact device for visualization and three-dimensional reconstruction. In this example, temporal multiplexing is used for alternatively providing a pattern 220 or a uniform illumination.

Because an optical fibre bundle 230 typically comprises several thousands of fibres, more than one monomode optical fibre 70 could be used for transmitting quasi-monchromatic light and forming a pattern 220 when the optical fibre bundle 230 includes monomode optical fibres. Every monomode optical fibre 70 can be considered as a single point source. Alternatively lighting different monomode optical fibres would result to induce different patterns 220 shifted with respect to one another. A first possibility to have such a device would be to have a laser source and a corresponding optical path for each of such monomode optical fibres. A second possibility would be to use one quasi-monochromatic light source that is directed to the entry of such different monomode optical fibres by using micro mirrors. By comparing different deformations of the patterns 220 induced by the different monomode optical fibres 70, one can expect to increase the spatial resolution of the three-dimensional reconstruction.

Also disclosed herein is an example method for visualization and three-dimensional reconstruction of an area of interest 200. The example method includes sending to the area of interest 200 a quasi-monochromatic light through a first cross-section 100 of at least one monomode optical fibre 70 and sending to the area of interest 200 light through a set of optical fibres 140 having a second cross-section 170. The example method also includes acquiring images of the area of interest 200 by using a camera 190 having a spatiotemporal resolution. In the example method, the at least one monomode optical fibre 70 and the set of optical fibres 140 are included in a same optical fibre bundle 230 of outer diameter D_{bundle}. Also, in the example method, a diffractive element 210 covers at least partially the second cross-section 170 of the set of optical fibres 140. Furthermore, in the example method, the spatiotemporal resolution of the camera 190 is such that the camera 190 is able to provide an image of a pattern 220 created by light emerging from the monomode optical fibre 70 and the diffractive element 210 on the area of interest 200, and able to provide a two-dimensional image of the area of interest 200 created by light emerging from the set of optical fibres 140 that appears uniformly illuminated. In some examples, the method further includes providing surgical tools that are connected to a tubular shell 20 comprising the optical fibre bundle 230.

In addition to the field of medical endoscopy, the example device 10 can be used in various applications. As an example, industrial endoscopes are used for inspecting anything hard to reach, such as jet engine interiors.

The teachings of present disclosure have been described in terms of specific examples and embodiments, which are illustrative of the teachings of the disclosure and not to be construed as limiting. More generally, it will be appreciated by persons skilled in the art that the present disclosure is not limited by what has been particularly shown and/or described hereinabove. Reference numerals in the claims do not limit their protective scope. Use of the verbs “to comprise”, “to include”, “to be composed of”, or any other variant, as well as their respective conjugations, does not exclude the presence of elements other than those stated. Use of the article “a”, “an” or “the” preceding an element does not exclude the presence of a plurality of such elements.

Summarized, the teachings of the present disclosure may also be described as follows. The example device 10 includes a first light source 60 able to send quasi-monochromatic light through a monomode optical fibre 70 and a second light source 130 able to send light through a set of optical fibres 140. A diffractive element 210 induces a pattern 220 to be projected on an area of interest 200 when the first light source 60 is switched on. A camera 190 has a spatiotemporal resolution such that it is able to visualize the pattern 220 created by the first light source 60 and the area of interest 200 illuminated by the second light source 130 that appears uniformly illuminated even if diffractive element 210 covers at least partially the second cross-section 170 of the set of optical fibres 140.

Although certain example methods and apparatus have been disclosed herein, the scope of coverage of this patent is not limited thereto. On the contrary, this patent covers all methods, apparatus and articles of manufacture fairly falling within the scope of the appended claims either literally or under the doctrine of equivalents.

1. A device comprising:
a tubular shell having a proximal end and a distal end;
a pattern projection optical group comprising:
a first light source that is quasi-monochromatic;
at least one monomode optical fibre positioned in the tubular shell, having a first extremity, a second extremity, and a first cross-section, and able to transport light through the first cross-section, the first extremity disposed at the proximal end, the second extremity disposed at the distal end; and
a first optical path between the first light source and the first extremity;
an illumination optical group comprising:
a second light source;
a set of optical fibres positioned in the tubular shell, the set of optical fibres having a third extremity and a fourth extremity and a second cross-section, the third extremity disposed at the proximal end, the fourth extremity disposed at the distal end; and
a second optical path between the second light source and the third extremity;
a diffractive element covering the first cross-section at the distal end; and
a camera having a spatiotemporal resolution;
wherein the at least one monomode optical fibre and the set of optical fibres are included in a same optical fibre bundle having an outer diameter D_{bundle};
wherein the diffractive element covers at least partially the second cross-section of the set of optical fibres at the fourth extremity; and
wherein the camera is to provide an image of a pattern created by the pattern projection optical group and the diffractive element on the area of interest and able to provide a two-dimensional image of the area of interest created by the illumination optical group that appears uniformly illuminated based on the spatiotemporal resolution of the camera.

2. The device according to claim 1, wherein the diffractive element covers at least between about 30% to about 70% of the second cross-section of the set of optical fibres at the fourth extremity.

3. The device according to claim 1, wherein the diffractive element totally covers the second cross-section of the set of optical fibres at the fourth extremity.

4. The device according to claim 1, wherein the illumination optical group is to provide incoherent light at the fourth extremity.

5. The device according to claim 1, wherein the camera has an outer diameter A_{cam} such that A_{cam}<2.4 D_{bundle}.

6. The device according to claim 1, wherein the camera has an outer diameter A_{cam} such that A_{cam}<0.6 D_{bundle}.

7. The device according to claim 1, wherein the area of interest has an outer diameter equal to φ, wherein the camera and the fourth extremity of the set of optical fibres are positioned at a same distance L from the area of interest, wherein the second light source is a quasi-monochromatic light source having a central wavelength equal to λ, and wherein the camera has a number of pixels along one direction, Nₗ, such that
$${N}_{l} < 2 \frac{φ}{L} \frac{{D}_{bundle}}{λ} .$$

8. The device according to claim 1, wherein the camera is positioned at the distal end.

9. The device according to claim 1, wherein the pattern projection optical group and the diffractive element are to provide an uncorrelated pattern on the area of interest.

10. The device according to claim 1, wherein multiplexing is used to distinguish a first image of a pattern created by the pattern projection optical group and the diffractive element from a second image created by the illumination optical group.

11. The device according to claim 10, wherein the multiplexing is a temporal multiplexing inducing light to be emitted from the first light source in a pulsed manner.

12. The device according to claim 1, wherein the set of optical fibres comprises multimode optical fibres.

13. The device according to claim 1, wherein the set of optical fibres comprises at least a hundred of monomode optical fibres.

14. The device according to claim 1 further comprising a third optical path between the second light source and the first extremity.

15. The device according to claim 1 further comprising channels in the tubular shell that have a geometry suitable for inserting tools for manipulating and cutting mammal tissues at the distal end.

16. A device comprising:
a tubular shell having a proximal end and a distal end;
a pattern projection optical group comprising:
a quasi-monochromatic light source;
at least one monomode optical fibre positioned in the tubular shell, having a first extremity, a second extremity, and a first cross-section, and able to transport light through the first cross-section, the first extremity disposed at the proximal end, the second extremity disposed at the distal end; and
a first optical path between the quasi-monochromatic light source and the first extremity;
an illumination optical group comprising:
the quasi-monochromatic light source;
a set of optical fibres positioned in the tubular shell, the set of optical fibres having a third extremity and a fourth extremity and a second cross-section, the third extremity disposed at the proximal end, the fourth extremity disposed at the at the distal end; and
a second optical path between the quasi-monochromatic light source and the third extremity;
a diffractive element covering the first cross-section at the distal end; and
a camera having a spatiotemporal resolution;
wherein the at least one monomode optical fibre and the set of optical fibres are included in a same optical fibre bundle having an outer diameter D_{bundle};
wherein the diffractive element covers at least partially the second cross-section of the set of optical fibres at the fourth extremity; and
wherein the camera is to provide an image of a pattern created by the pattern projection optical group and the diffractive element on the area of interest and able to provide a two-dimensional image of the area of interest created by the illumination optical group that appears uniformly illuminated based on the spatiotemporal resolution of the camera.

17. The device according to claim 16, wherein the camera has an outer diameter A_{cam} such that A_{cam}<2.4 D_{bundle}.

18. The device according to claim 16, wherein the area of interest has an outer diameter equal to φ, wherein the camera and the fourth extremity of the set of optical fibres are positioned at a same distance L from the area of interest, wherein the quasi-monochromatic light source has a central wavelength equal to λ, and wherein the camera has a number of pixels along one direction, Nₗ, such that
$${N}_{l} < 2 \frac{φ}{L} \frac{{D}_{bundle}}{λ} .$$

19. A method comprising:
sending to an area of interest a quasi-monochromatic light through a first cross-section of at least one monomode optical fibre;
sending to the area of interest light through a set of optical fibres having a second cross-section, wherein the at least one monomode optical fibre and the set of optical fibres are included in a same optical fibre bundle of outer diameter D_{bundle}, and wherein a diffractive element covers at least partially the second cross-section of the set of optical fibres;
acquiring images of the area of interest by using a camera having a spatiotemporal resolution; and
providing, using the camera, (1) an image of a pattern created by light emerging from the monomode optical fibre and the diffractive element on the area of interest, and (2) a two-dimensional image of the area of interest created by light emerging from the set of optical fibres that appears uniformly illuminated.

20. The method according to claim 19 further comprising providing surgical tools that are connected to a tubular shell comprising the optical fibre bundle.
